(19) United States
(12) Patent Application Publication
(10) Pub. No.: US 2003/0017975 A1
(43) Pub. Date: Jan. 23, 2003

(54) Use

(76) Inventors: Fumiaki Ikeda, Osaka (JP); Kazumi Otomo, Osaka (JP); Satoru Matsumoto, Osaka (JP); Yoshimi Wakai, Osaka (JP); Noriko Teratani, Osaka (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/148,876

(22) PCT Filed: Dec. 8, 2000
(86) PCT No.: PCT/JP00/08730

(30) Foreign Application Priority Data
Dec. 13, 1999 (AU) PQ 4623

(51) Int. Cl. A61K 38/12; A61K 38/13

(52) U.S. Cl. 514/9

(57) ABSTRACT

The present invention is relating to a new use of an immunosuppressant, for increasing an antifungal activity of a lipopeptide compound.

### TECHNICAL FIELD

This invention relates to a new use of an immunosuppressant, which is useful in a medical field.

### BACKGROUND ART

Various immunosuppressants are well known. And, a certain lipopeptide compound, and its related compounds are known to have an antifungal activity and inhibit an activity of β-1,3-glucan synthase (WO96/11210 and WO99/40108).

### DISCLOSURE OF INVENTION

This invention relates to a new use of an immunosuppressant, for increasing an antifungal activity of a lipopeptide compound.

Therefore, one object of the present invention is to provide a new use of an immunosuppressant for increasing an antifungal activity of a lipopeptide compound.

Another object of this invention is to provide a method for increasing an antifungal activity of a lipopeptide compound by administering an effective amount of an immunosuppressant.

A further object of this invention is to provide a use of an immunosuppressant for manufacturing a medicament for increasing an antifungal activity of a lipopeptide compound.

Still further object of this invention is to provide a composition comprising an immunosuppressant, for increasing an antifungal activity of a lipopeptide compound.

Preferable “immunosuppressant” is, for example, the tricyclic macrolide shown in EP-0184162, WO89/05303, WO93/05058, WO96/31514, and so on, the disclosure of which is incorporated herein by reference.

As a particular example of the tricyclic macrolides compounds, the tricyclic compound of the following formula (II) can be exemplified.

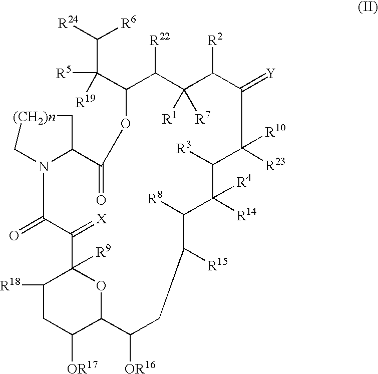

(wherein each of adjacent pairs of R¹ and R² R³ and R⁴, and R⁵ and R⁶ independently

(a) is two adjacent hydrogen atoms, but R² may also be an alkyl group or

(b) may form another bond formed between the carbon atoms to which they are attached;

R⁷ is a hydrogen atom, a hydroxy group, a protected hydroxy group, or an alkoxy group, or an oxo group together with R¹;

R⁸ and R⁹ are independently a hydrogen atom or a hydroxy group;

R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;

X is an oxo group, (a hydrogen atom and a hydroxy group), (a hydrogen atom and a hydrogen atom), or a group represented by the formula —CH₂O—;

Y is an oxo group, (a hydrogen atom and a hydroxy group), (a hydrogen atom and a hydrogen atom), or a group represented by the formula N—NR¹¹R¹² or N—OR¹³;

R¹¹ and R¹² are independently a hydrogen atom, an alkyl group, an aryl group or a tosyl group;

R¹³, R¹⁴, R¹⁵, R¹⁶, R¹⁷, R¹⁸, R¹⁹, R²² and R²³ are independently a hydrogen atom or an alkyl group;

R²⁴ is an optionally substituted ring system which may contain one or more heteroatoms;

n is an integer of 1 or 2; and

in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se(C₆H₅), and an alkyl substituted by one or more hydroxy groups.

The definitions used in the above general formula (II) and the specific and preferred examples thereof are now explained and set forth in detail.

The term “lower” means, unless otherwise indicated, a group having 1 to 6 carbon atoms.

Preferable examples of the “alkyl groups” and an alkyl moiety of the “alkoxy group” include a straight or branched chain aliphatic hydrocarbon residue, for example, a lower alkyl group such as methyl, ethyl, propyl, isopropyl, butyl, isobutyl, pentyl, neopentyl and hexyl.

Preferable examples of the “alkenyl groups” include a straight or branched chain aliphatic hydrocarbon residue having one double-bond, for example, a lower alkenyl group such as vinyl, propenyl (e.g., allyl group), butenyl, methylpropenyl, pentenyl and hexenyl.

Preferable examples of the “aryl groups” include phenyl, tolyl, xylyl, cumenyl, mesityl and naphthyl.

Preferable protective groups in the “protected hydroxy groups” and the “protected amino” are 1-(lower alkylthio)-(lower)alkyl group such as a lower alkylthiomethyl group (e.g., methylthiomethyl, ethylthiomethyl, propylthiomethyl, isopropylthiomethyl, butylthiomethyl, isobutylthiomethyl, hexylthiomethyl, etc.), more preferably C₁-C₄ alkylthiomethyl group, most preferably methylthiomethyl group;

trisubstituted silyl group such as a tri(lower)alkylsilyl (e.g., trimethylsilyl, triethylsilyl, tributylsilyl, tert-butyldimethylsilyl, tri-tert-butylsilyl, etc.) or lower alkyl-diarylsilyl (e.g., methyldiphenylsilyl, ethyldiphenylsilyl, propyldiphenylsilyl, tert-butyldiphenyl-silyl, etc.), more preferably tri(C₁-C₄)alkylsilyl group and C₁-C₄ alkyldiphenylsilyl group, most preferably tert-butyldimethylsilyl group and tert-butyldiphenylsilyl group; and an acyl group such as an aliphatic, aromatic acyl group or an aliphatic acyl group substituted by an aromatic group, which are derived from a carboxylic acid, sulfonic acid or carbamic acid.

Examples of the aliphatic acyl groups include a lower alkanoyl group optionally having one or more suitable substituents such as carboxy, e.g., formyl, acetyl, propionyl, butyryl, isobutyryl, valeryl, isovaleryl, pivaloyl, hexanoyl, carboxyacetyl, carboxypropionyl, carboxybutyryl, carboxyhexanoyl, etc.; a cyclo(lower)alkoxy(lower)alkanoyl group optionally having one or more suitable substituents such as lower alkyl, e.g., cyclopropyloxyacetyl, cyclobutyloxypropionyl, cycloheptyloxybutyryl, menthyloxyacetyl, menthyloxypropionyl, menthyloxybutyryl, menthyloxypentanoyl, menthyloxyhexanoyl, etc.; a camphorsulfonyl group; or a lower alkylcarbamoyl group having one or more suitable substituents such as carboxy or protected carboxy, for example, carboxy(lower)alkylcarbamoyl group (e.g., carboxymethylcarbamoyl, carboxyethylcarbamoyl, carboxypropylcarbamoyl, carboxybutylcarbamoyl, carboxypentylcarbamoyl, carboxyhexylcarbamoyl, etc.), tri-(lower)alkylsilyl(lower)alkoxycarbonyl(lower)alkylcarbamoyl group (e.g., trimethylsilylmethoxycarbonylethylcarbamoyl, trimethylsilylethoxycarbonylpropylcarbamoyl, triethylsilylethoxycarbonylpropylcarbamoyl, tert-butyldimethylsilylethoxycarbonylpropylcarbamoyl, tri-methylsilylpropoxycarbonylbutylcarbamoyl, etc.) and so on.

Examples of the aromatic acyl groups include an aroyl group optionally having one or more suitable substituents such as nitro, e.g., benzoyl, toluoyl, xyloyl, naphthoyl, nitrobenzoyl, dinitrobenzoyl, nitronaphthoyl, etc.; and an arenesulfonyl group optionally having one or more suitable substituents such as halogen, e.g., benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl, etc.

Examples of the aliphatic acyl groups substituted by an aromatic group include ar(lower)alkanoyl group optionally having one or more suitable substituents such as lower alkoxy or trihalo(lower)alkyl, e.g., phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl, etc.

More preferable acyl groups among the aforesaid acyl groups are C₁-C₄ alkanoyl group optionally having carboxy, cyclo(C₅-C₆)alkoxy(C₁-C₄)alkanoyl group having two (C₁-C₄) alkyls at the cycloalkyl moiety, camphorsulfonyl group, carboxy-(C₁-C₄)alkylcarbamoyl group, tri (C₁-C₄) alkylsilyl (C₁-C₄) alkoxycarbonyl (C₁-C₄)-alkylcarbamoyl group, benzoyl group optionally having one or two nitro groups, benzenesulfonyl group having halogen, or phenyl(C₁-C₄)alkanoyl group having C₁-C₄ alkoxy and trihalo(C₁-C₄) alkyl group. Among these, the most preferable ones are acetyl, carboxypropionyl, menthyloxyacetyl, camphorsulfonyl, benzoyl, nitrobenzoyl, dinitrobenzoyl, iodobenzenesulfonyl and 2-trifluoromethyl-2-methoxy-2-phenylacetyl.

Preferable examples of the “5- or 6-membered nitrogen, sulfur and/or oxygen containing heterocyclic ring” include a pyrrolyl group and a tetrahydrofuryl group.

R²⁴ is an optionally substituted ring system which may contain one or more heteroatoms, Preferable R²⁴ may be cyclo (C₅₋₇) alkyl group optionally having suitable substituents, and the following ones can be exemplified.

(a) a 3,4-di-oxo-cyclohexyl group;

(b) a 3-R²⁰-4-R²¹-cyclohexyl group,

in which

R²⁰ is hydroxy, an alkoxy group, an oxo group, or a —OCH₂OCH₂CH₂OCH₃ group, and

R²¹ is hydroxy, —OCN, an alkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,

in which

R²⁵ is optionally protected hydroxy or protected amino, and

R²⁶ is hydrogen or methyl, or

R²⁰ and R²¹ together form an oxygen atom in an epoxide ring; or

(c) cyclopentyl group substituted by methoxymethyl, optionally protected hydroxymethyl, acyloxymethyl

(in which the acyl moiety optionally contains either a dimethylamino group which may be quaternized, or a carboxy group which may be esterified), one or more amino and/or hydroxy groups which may be protected, or aminooxalyloxymethyl. A preferred example is a 2-formyl-cyclopentyl group.

“A heteroaryl which may be substituted by suitable substituents” moiety of the “heteroaryloxy which may be substituted by suitable substituents” may be the ones exemplified for R¹ of the compound of the formula of EP-A-532,088, with preference given to 1-hydroxyethylindol-5-yl, the disclosure of which is incorporated herein by reference.

The tricyclic compounds (II) and its pharmaceutically acceptable salt for use in accordance with this invention are well known to have excellent immunosuppressive activity, antimicrobial activity and other pharmacological activities and, as such, be of value for the treatment or prevention of rejection reactions by transplantation of organs or tissues, graft-vs-host diseases, autoimmune diseases, and infectious diseases [EP-A-0184162, EP-A-0323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/04680, WO93/5059, etc.], the disclosures of which are incorporated herein by reference.

Particularly, the compounds which are designated as FR900506 (=FK506), FR900520 (ascomycin), FR900523, and FR900525 are products produced by microorganisms of the genus Streptomyces, such as Streptomyces tsukubaensis No. 9993 [deposited with National Institute of Bioscience and Human Technology Agency of Industrial Science and Technology (formerly Fermentation Research Institute Agency of Industrial Science and Technology ), at 1-3, Higashi 1-chome, Tsukuba-shi, Ibaraki, Japan, date of deposit Oct. 5, 1984, accession number FERM BP-927] or Streptomyces hygroscopicus subsp. yakushimaensis No. 7238 [deposited with National Institute of Bioscience and Human Technology Agency of Industrial Science and Technology (formerly Fermentation Research Institute Agency of Industrial Science and Technology ), at 1-3, Higashi 1-chome, Tsukuba-shi, Ibaraki, Japan, date of deposit Jan. 12, 1985, accession number FERM BP-928][EP-A-0184162]. The FK506 (general name: tacrolimus) of the following chemical formula, in particular, is a representative compound.

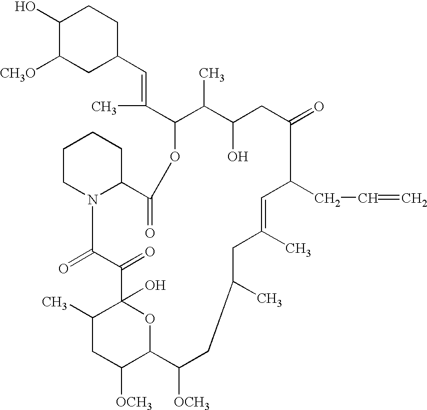

Chemical name: 17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone

The preferred examples of the tricyclic compounds (II) are the ones, wherein each of adjacent pairs of R³ and R⁴ or Rs and R⁶ independently form another bond formed between the carbon atoms to which they are attached;

each of R⁸ and R²³ is independently a hydrogen atom;

R⁹ is a hydroxy group;

R¹⁰ is a methyl group, an ethyl group, a propyl group or an allyl group;

X is (a hydrogen atom and a hydrogen atom) or an oxo group;

Y is an oxo group;

each of R¹⁴ R¹⁵, R¹⁶, R¹⁷, R¹⁸, R¹⁹, and R²² is a methyl group;

R²⁴ is a 3-R²⁰-4-R²¹-cyclohexyl group,

in which

R²⁰ is hydroxy, an alkoxy group, an oxo group, or a —OCH₂OCH₂CH₂OCH₃ group, and

R²¹ is hydroxy, —OCN, an alkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHOO—

in which

R²⁵ is optionally protected hydroxy or protected amino, and

R²⁶ is hydrogen or methyl, or

R²⁰ and R²¹ together form an oxygen atom in an epoxide ring; and

n is an integer of 1 or 2.

The most preferable tricyclic compounds (II) is, in addition to FK506, ascomycin derivatives such as halogenated-ascomycin (e.g., 33-epi-chloro-33-desoxyascomycin), which is disclosed in EP 427,680, example 66a.

As the other preferable example of the immunosuppressant, rapamycin [THE MERCK INDEX (12th edition), No. 8288] and its derivatives can be exemplified. Preferred example of the derivatives is an O-substituted derivative in which the hydroxy in position 40 of formula A illustrated at page 1 of WO 95/16691, incorporated herein by reference, is replaced by —OR₁ in which R₁ is hydroxyalkyl, hydroalkoxyalkyl, acylaminoalkyl and aminoalkyl; for example 40-O-(2-hydroxy)ethyl-rapamycin, 40-O-(3-hydroxy)propyl-rapamycin, 40-O-[2-(2-hydroxy)ethoxy]ethyl-rapamycin and 40-O-(2-acetaminoethyl)-rapamycin. These O-substituted derivatives may be produced by reacting rapamycin (or dihydro or deoxo-rapamycin) with an organic radical attached to a leaving group (for example RX where R is the organic radical which is desired as the O-substituent, such as an alkyl, allyl, or benzyl moiety, and X is a leaving group such as CCl₃C(NH)O or CF₃SO₃) under suitable reaction conditions. The conditions may be acidic or neutral conditions, for example in the presence of an acid like trifluoromethanesulfonic acid, camphorsulfonic acid, p-toluenesulfonic acid or their respective pyridinium or substituted pyridinium salts when X is CCl₃C(NH)O or in the presence of a base like pyridine, a substituted pyridine, diisopropylethylamine or pentamethylpiperidine when X is CF₃SO₃. The most preferable one is 40-O-(2-hydroxy) ethyl rapamycin, which is disclosed in WO94/09010, the disclosure of which is incorporated herein by reference.

The tricyclic compound(II), and rapamycin and its derivatives, may be in a form of its salt, which includes conventional non-toxic and pharmaceutically acceptable salt such as the salt with inorganic or organic bases, specifically, an alkali metal salt such as sodium salt and potassium salt, an alkali earth metal salt such as calcium salt and magnesium salt, an ammonium salt and an amine salt such as triethylamine salt and N-benzyl-N-methylamine salt.

With respect to the immunosuppressant usable in the present invention, particularly the tricyclic macrolide compounds, it is to be understood that there may be conformers and one or more stereoisomers such as optical and geometrical isomers due to asymmetric carbon atom(s) or double bond(s), and such conformers and isomers are also included within the scope of the present invention. And further, the tricyclic macrolide compounds can be in the form of a solvate, which is included within the scope of the present invention. The solvate preferably include a hydrate and an ethanolate.

Further example of the immunosuppressant is cyclosporin and its derivatives such as cyclosporin A, B, C, D, E, F, G, etc, which are shown in THE MERCK INDEX (12th edition), No. 2821, U.S. Pat. Nos. 4,117,118, 4,215,199, 4,288,431, 4,388,307, Helv. Chim. Acta. 60, 1568(1977) and 65, 1655(1982), Transplant. Proc. 17, 1362(1985), and so on. Among which, the most preferable one is cyclosporin A. The disclosures of the above references are incorporated herein.

In the present invention, the lipopeptide compound is shown by the following formula(I).

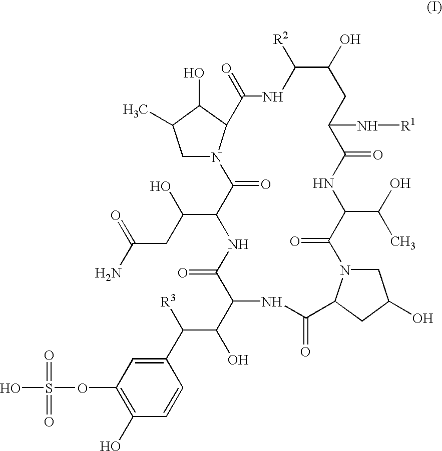

Wherein R¹ is acyl group,

R² is hydrogen or hydroxy and

R³ is hydrogen or hydroxy,

or a salt thereof.

Suitable salt of the lipopeptide compound(I) is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt; a salt with an organic base, for example, an organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride hydrobromide, sulfate, phosphate, etc.);

an organic carboxylic sulfonic acid addition salt (e.g., formate, acetate, trifluoroacetate, maleate, tartrate, fumarate, methanesulfonate, benzenesulfonate, toluenesulfonate, etc.); a salt with a basic or acidic amino acid (e.g., arginine, aspartic acid, glutamic acid, etc.).

It is to be noted that each of the lipopeptide compound(I) may include one or more stereoisomer(s) such as optical isomer(s) and geometrical isomer(s) due to asymmetric carbon atom(s) and double bond(s), and all such isomer(s) and the mixture thereof are included within the scope of the present invention.

The lipopeptide compound(I) or a salt thereof includes solvated compound [e.g., hydrate, ethanolate, etc.)].

The lipopeptide compound(I) or a salt thereof includes both its crystal form and non-crystal form.

It should be understood that the lipopeptide compound(I) in the present invention may include the prodrug form.

In the lipopeptide compound(I), suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);

lower or higher alkoxycarbonyl (e.g., methoxycarbonyl, ethoxycarbonyl, t-butoxycarbonyl, t-pentyloxycarbonyl, heptyloxycarbonyl, etc.);

lower or higher alkylsulfonyl (e.g., methylsulfonyl, ethylsulfonyl, etc.);

lower or higher alkoxysulfonyl (e.g., methoxysulfonyl, ethoxysulfonyl, etc.); or the like;

Aromatic acyl such as

aroyl (e.g., benzoyl, toluoyl, naphthoyl, etc.); aroyl which has one or more suitable substituent(s); ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];

ar(lower)alkenoyl [e.g., phenyl(C₃-C₆)alkenoyl (e.g., phenylpropenoyl, phenylbutenoyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.];

ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.];

aryloxycarbonyl (e.g., phenoxycarbonyl, naphthyloxycarbonyl, etc.);

aryloxy(lower)alkanoyl (e.g., phenoxyacetyl, phenoxypropionyl, etc.);

arylcarbamoyl (e.g., phenylcarbamoyl, etc.);

arylthiocarbamoyl (e.g., phenylthiocarbamoyl, etc.);

arylglyoxyloyl (e.g., phenylglyoxyloyl, naphthylglyoxyloyl, etc.);

arylsulfonyl which may have 1 to 4 lower alkyl (e.g., phenylsulfonyl, p-tolylsulfonyl, etc.); or the like;

Heterocyclic acyl such as

heterocycliccarbonyl; heterocyclic(lower)alkanoyl (e.g., heterocyclicacetyl, heterocyclicpropanoyl, heterocyclicbutanoyl, heterocyclicpentanoyl, heterocyclichexanoyl, etc.);

heterocyclic(lower)alkenoyl (e.g., heterocyclicpropenoyl, heterocyclicbutenoyl, heterocyclicpentenoyl, heterocyclichexenoyl, etc.);

heterocyclicglyoxyloyl; or the like.

Among them, more preferred “acyl group” is aroyl which has one or more suitable substituent(s).

Suitable example of “suitable substituent(s)” in the term of “aroyl which has one or more suitable substituent(s)” may be heterocyclic group substituted with aryl having lower alkoxy, heterocyclic group substituted with aryl having lower alkoxy(lower)alkoxy, heterocyclic group substituted with aryl having lower alkoxy(higher)alkoxy, heterocyclic group substituted with aryl having cyclo(lower)alkyloxy, heterocyclic group substituted with aryl having heterocyclic group, heterocyclic group substituted with cyclo(lower)alkyl having cyclo(lower)alkyl, heterocyclic group substituted with aryl having aryl substituted with lower alkoxy(lower)alkoxy, heterocyclic group substituted with aryl having heterocyclic group substituted with cyclo(lower)alkyl;

in which the preferred one may be unsaturated 3 to 8-membered heteromonocyclic group containing 1 to 2 oxygen atom(s) and 1 to 3 nitrogen atom(s) substituted with phenyl having (C₄-C₆)alkoxy, unsaturated condensed heterocyclic group containing 1 or 2 sulfur atom(s) and 1 to 3 nitrogen atom(s) substituted with phenyl having (C₄-C₆)alkoxy, unsaturated 3 to 8-membered heteromonocyclic group containing 1 or 2 sulfur atom(s) and 1 to 3 nitrogen atom(s) substituted with phenyl having (C₁-C₄)alkoxy(C₄-C₆)alkoxy, unsaturated 3 to 8-membered heteromonocyclic group containing 1 or 2 sulfur atom(s) and 1 to 3 nitrogen atom(s) substituted with phenyl having (C₁-C₄)alkoxy(C₇-C₁₄)alkoxy, saturated 3 to 8-membered heteromonocyclic group containing 1 to 4 nitrogen atom(s) substituted with phenyl having (C₁-C₄)alkoxy(C₇-C₁₄)alkoxy, unsaturated condensed heterocyclic group containing 1 or 2 sulfur atom(s) and 1 to 3 nitrogen atom(s) substituted with phenyl having cyclo(C₄-C₆)alkyloxy, unsaturated condensed heterocyclic group containing 1 or 2 sulfur atom(s) and 1 to 3 nitrogen atom(s) substituted with phenyl saturated 3 to 8-membered heteromonocyclic group containing 1 or 2 oxygen atom(s) and 1 to 3 nitrogen atom(s), saturated 3 to 8-membered heteromonocyclic group containing 1 to 4 nitrogen atom(s) substituted with cyclo(C₄-C₆)alkyl having cyclo(C₄-C₆)alkyl, unsaturated 3 to 8-membered heteromonocyclic group containing 1 or 2 sulfur atom(s) and 1 to 3 nitrogen atom(s) substituted with phenyl having phenyl substituted with (C₁-C₄)alkoxy(C₁-C₄)alkoxy, unsaturated 3 to 8-membered heteromonocyclic group containing 1 or 2 sulfur atom(s) and 1 to 3 nitrogen atom(s) substituted with phenyl having saturated 3 to 8-membered heteromonocyclic group containing 1 to 4 nitrogen atom(s) substituted with cyclo(C₄-C₆)alkyl, unsaturated condensed heterocyclic group containing 1 or 2 sulfur atom(s) and 1 to 3 nitrogen atom(s) substituted with phenyl having saturated 3 to 8-membered heteromonocyclic group containing 1 to 4 nitrogen atom(s) having cyclo(C₄-C₆)alkyl, etc.

Among them, the most preferred one may be isoxazolyl substituted with phenyl having pentyloxy, imidazothiadiazolyl substituted with phenyl having pentyloxy, thiadiazolyl substituted with phenyl having methoxyhexyloxy, thiadiazolyl substituted with phenyl having methoxyoctyloxy, thiadiazolyl substituted with phenyl having methoxyheptyloxy, imidazothiadiazolyl substituted with phenyl having cyclohexyloxy, imidazothiadiazolyl substituted with phenyl having dimethylmorpholino, piperazinyl substituted with phenyl having methoxyheptyloxy, piperazinyl substituted with phenyl having methoxyoctyloxy, piperazinyl substituted with cyclohexyl having cyclohexyl, thiadiazolyl substituted with phenyl having phenyl substituted with methoxyethoxy, thiadiazolyl substituted with phenyl having phenyl substituted with methoxybutoxy, thiadiazolyl substituted with phenyl having phenyl substituted with ethoxypropoxy, imidazothiadiazolyl substituted with phenyl having piperazinyl substituted with cyclohexyl, imidazothiadiazolyl substituted with phenyl having piperazinyl substituted with cyclohexyl.

The more suitable example of “acyl group” of R¹ may be benzoyl which has isoxazolyl substituted with phenyl having pentyloxy, benzoyl which has imidazolthiadiazolyl substituted with phenyl having pentyloxy, benzoyl which has thiadiazolyl substituted with phenyl having methoxyhexyloxy, benzoyl which has thiadiazolyl substituted with phenyl having methoxyoctyloxy, benzoyl which has thiadiazolyl substituted with phenyl having methoxyheptyloxy, benzoyl which has imidazothiadiazolyl substituted with phenyl having cyclohexyloxy, benzoyl which has imidazothiadiazolyl substituted with phenyl having dimethylmorpholino, benzoyl which has piperazinyl substituted with phenyl having methoxyheptyloxy, benzoyl which has piperazinyl substituted with phenyl having methoxyoctyloxy, benzoyl which has piperazinyl substituted with cyclohexyl having cyclohexyl, benzoyl which has thiadiazolyl substituted with phenyl having phenyl substituted with methoxyethoxy, benzoyl which has thiadiazolyl substituted with phenyl having phenyl substituted with methoxybutoxy, benzoyl which has thiadiazolyl substituted with phenyl having phenyl substituted with ethoxypropoxy, benzoyl which has imidazothiadiazolyl substituted with phenyl having piperazinyl substituted with cyclohexyl, benzoyl which has imidazothiadiazolyl substituted with phenyl having piperazinyl substituted with cyclohexyl.

In the present invention, an immunosuppressant is able to be administered for increasing an antifungal activity of the lipopeptide compound(I) simultaneously, separately or in sequential use with the lipopeptide compound(I).

If advisable, the lipopeptide compound(I) can be mixed with the immunosuppressant prior to its use. So, the composition comprising the said the immunosuppressant of the present invention may further comprise the lipopeptide compound(I). And optionally, it comprises further additional ingredients, such as, mycophenolate mofetil (CellCept), steroids, Azathiopurine, and so on.

While the effective dosage of the immunosuppressant depends on the type of the said immunosuppressant, the patient's age, type of disease, severity of illness, and other factors, a daily dose thereof is about 0.01˜1000 mg, preferably 0.05˜500 mg, and more preferably, 0.1˜100 mg for therapeutic purposes. The average unit dose may be generally about 0.1 mg, 0.5 mg, 1 mg, 5 mg, 10 mg, 50 mg, 100 mg, 250 mg, or 500 mg.

The pharmaceutical composition of the immunosuppressant can be used in the form of a pharmaceutical preparation, for example, in solid, semisolid or liquid form, which contains the immunosuppressant or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The lipopeptide compound(I) or a pharmaceutically acceptable salt thereof may also be included in the pharmaceutical composition of the present invention in an amount sufficient to produce the desired antimicrobial effect upon the process or condition of diseases.

For applying the lipopeptide compound(I) or a salt thereof to human, it is preferable to apply it by intravenous, intramuscular, pulmonary, oral administration, or insufflation. While the dosage of therapeutically effective amount of the lipopeptide compound(I) varies from and also depends upon the age and condition of each individual patient to be treated, in the case of intravenous administration, a daily dose of 0.01-20 mg of the lipopeptide compound(I) per kg weight of human being in the case of intramuscular administration, a daily dose of 0.1-20 mg of the lipopeptide compound(I) per kg weight of human being, in case of oral administration, a daily dose of 0.5-50 mg of the lipopeptide compound(I) per kg weight of human being is generally given for treating or preventing infectious diseases.

Especially in case of the treatment of prevention of Pneumocystis carinii infection, the followings are to be noted.

For administration by inhalation, the lipopeptide compound(I) is conveniently delivered in the form of an aerosol spray presentation from pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

The lipopeptide compound(I) or a salt thereof has antifungal activities, in which the fungi may include Acremonium;

Absidia (e.g., Absidia corymbifera, etc);

Aspergillus (e.g., Aspergillus clavatus, Aspergillus flavus, Aspergillus fumigatus, Aspergillus nidulans, Aspergillus niger, Aspergillus terreus, Aspergillus versicolor, etc);

Blastomyces (e.g., Blastomyces dermatitidis, etc);

Candida (e.g., Candida albicans, Candida glabrata, Candida guilliermondii, Candida kefyr, Candida krusei, Candida parapsilosis, Candida stellatoidea, Candida tropicalis, Candida utilis, etc.);

Cladosporium (e.g., Cladosporium trichoides, etc);

Coccidioides (e.g., Coccidioides immitis, etc);

Cryptococcus (e.g., Cryptococcus neoformans, etc);

Cunninghamella (e.g., Cunninghamella elegans, etc);

Dermatophyte;

Exophiala (e.g., Exophiala dermatitidis, Exophiala spinifera, etc);

Epidermophyton (e.g., Epidermophyton floccosum, etc);

Fonsecaea (e.g., Fonsecaea pedrosoi, etc);

Fusarium (e.g., Fusarium solani, etc);

Geotrichum (e.g., Geotrichum candiddum, etc);

Histoplasma (e.g., Histoplasma capsulatum var. capsulatum, etc);

Malassezia (e.g., Malassezia furfur, etc);

Microsporum (e.g., Microsporum canis, Microsporum gypseum, etc);

Mucor;

Paracoccidioides (e.g., Paracoccidioides brasiliensis, etc);

Penicillium (e.g., Penicillium marneffei, etc);

Phialophora;

Pneumocystis (e.g., Pneumocystis carinii, etc);

Pseudallescheria (e.g., Pseudallescheria boydii, etc);

Rhizopus (e.g., Rhizopus microsporus var. rhizopodiformis, Rhizopus oryzae, etc);

Saccharomyces (e.g., Saccharomyces cerevisiae, etc);

Scopulariopsis;

Sporothrix (e.g., Sporothrix schenckii, etc);

Trichophyton (e.g., Trichophyton mentagrophytes, Trichophyton rubrum, etc);

Trichosporon (e.g., Trichosporon asahii, Trichosporon cutaneum, etc).

The above fungi are well known to cause various infection diseases in skin, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph duct, gastrointestine, articulation, muscle, tendon, interstitial plasma cell in lung, and so on.

Therefore, the composition of the present invention are useful for preventing and treating various infectious diseases, such as dermatophytosis(e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, and so on.

Particularly, the lipopeptide compound(I) or a salt thereof has an inhibitory activity on β-1,3-glucan synthase, and therefore which are expected to be useful for the prophylactic and/or therapeutic treatment of Pneumocystis carinii infection (e.g. Pneumocystis carinii pneumonia) in a human being or an animal (WO96/11210 and WO99/40108). Therefore, the present composition is useful for prophylactic and/or therapeutic treatment of infectious diseases caused by the above-mentioned fungi.

From the another aspect, the present invention also provides the following inventions.

i) A use of an immunosuppressant for manufacturing a medicament for increasing an effect caused by a lipopeptide compound, in which the effect caused by a lipopeptide compound is an antifungal activity.

ii) A use of an immunosuppressant and a lipopeptide compound for manufacturing a medicament for simultaneous, separate or sequential use for an antifungal activity.

iii) A method for increasing an antifungal activity of a lipopeptide compound, by administering an effective amount of immunosuppressant to a human being or an animal.

iv) A method for preventing and treating infectious diseases, by administering an effective amount of a lipopeptide compound and an effective amount of an immunosuppressant to a human being or an animal.

v) A composition comprising an immunosuppressant for increasing an antifungal activity of a lipopeptide compound.

vi) A composition comprising an immunosuppressant and a lipopeptide compound as a combined preparation for simultaneous, separate or sequential use for an antifungal activity.

vii) An article of manufacture, comprising packaging material and the composition mentioned in the above v) or vi) contained within said the composition is therapeutically effective for infectious diseases, and wherein said packaging material comprises a label or a written material which indicates that said composition can be used for infectious diseases.

viii) A commercial package comprising the composition mentioned in the above v) or vi), and a written matter associated therewith, wherein the written matter states that the composition can or should be used for preventing or treating infectious diseases.

The invention is further illustrated in connection with the following non-limiting example.

Test Compounds

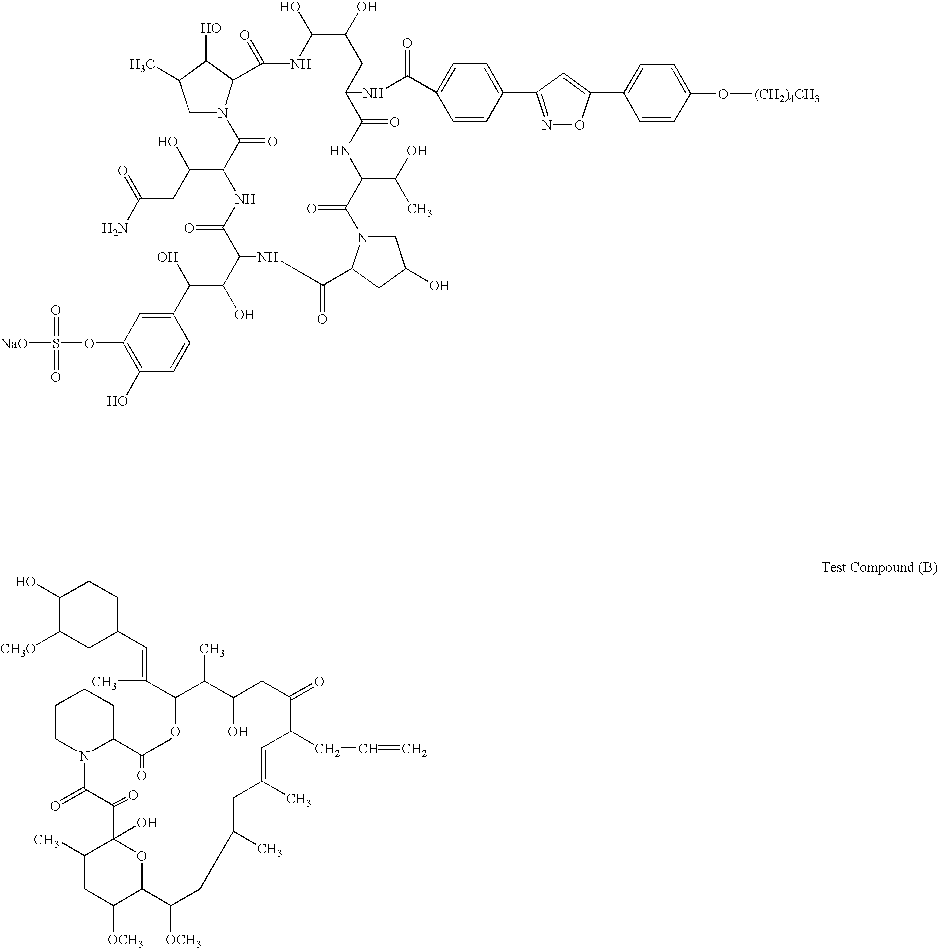

Test Method

The broth microdilloution method using RPMI medium (pH7.0) was used, comparing the each drug alone (Test Compound (A) and Test Compound (B)) and combined for clinical isolate of Aspergillus fumigatus. A combination of drug concentrations was evaluated by the checkerboared method.

All wells were examined macroscopically for growth and compared to a control (no drug). MIC was visually determined as the lowest concentration resulting in prominent decrease in turbidity compared to controls.

The Fractional Inhibitory Concentration(FIC) for each drug in mixture wells was compared to the MIC for each drug alone. The FIC index was calculated from the sum of the FICs for the two drugs. A quantitative expression of the interaction for inhibition is as follows:

Synergy=<0.5;

Test Result

In vitro combination with Test Compound (A) and Test Compound (B) against A. fumigatus

| | MIC (μg/ml) | | | | |
|---|---|---|---|---|---|
| | | Test | | Test | |
| | Test | Com- | Test | Com- | |
| | Com- | pound | Com- | pound (B) | |
| | pound (A) | Com- | pound (B) | com- | |
| Organism | Alone | bination | Alone | bination | FIC index |
| A. fumigatus | 0.0156 | 0.0039 | 0.125 | 0.0313 | 0.50 |
| FP1996 | | | | | |

From the results of the above example, synergy effect of efficacy was observed with combination of the lipopeptide compound(I) and the immunosuppressant at certain concentrations. No antagonism of efficacy with the immunosuppressant in combination with the lipopeptide compound(I) also was seen.

Given the above disclosure, it is thought that variations will occur to those skilled in the art. For example, it is thought that combination using other immunosuppressant and the lipopeptide compounds other than the lipopeptide compound(I) may also be effective against fungal infections caused by the fungal pathogens noted. Accordingly, it is intended that the above example should be construed as illustrative and that the invention disclosed herein should be limited only by the following claims.

The patents, patent applications and publications cited herein are incorporated by reference.

1. A use of an immunosuppressant for manufacturing a medicament for increasing an antifungal activity of a lipopeptide compound.

2. The use of the claim 1, in which the lipopeptide compound inhibits the production of β-1,3-glucan.

3. The use of the claim 1, in which the lipopeptide compound inhibits the activity of β-1,3-glucan synthase.

4. The use of the claim 1, in which the immunosuppressant is tacrolimus or its hydrate, or cyclosporins, and the lipopeptide compound is shown by the following formula(I): 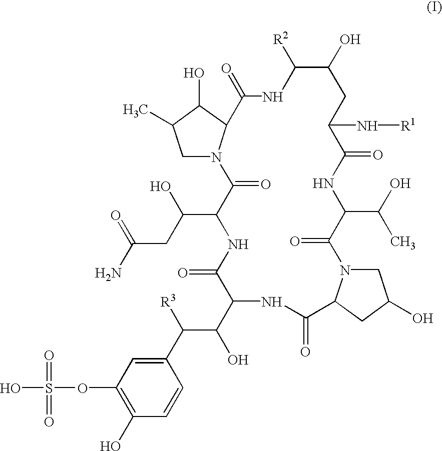
Wherein R¹ is acyl group,
R² is hydrogen or hydroxy and
R³ is hydrogen or hydroxy,
or a salt thereof.

5. A use of an immunosuppressant and a lipopeptide compound for manufacturing a medicament for simultaneous, separate or sequential use for an antifungal activity.

6. A method for increasing an antifungal activity of a lipopeptide compound, by administering an effective amount of an immunosuppressant to a human being or an animal.

7. A method for preventing and treating infectious diseases, by administering an effective amount of an immunosuppressant and an effective amount of a lipopeptide compound to a human being or an animal.

8. A composition comprising an immunosuppressant, for increasing an antifungal activity of a lipopeptide compound.

9. A composition comprising an immunosuppressant and a lipopeptide compound as a combined preparation for simultaneous, separate or sequential use for an antifungal activity.

10. An article of manufacture, comprising packaging material and the composition claimed in claim 8 or 9 contained within said packaging material, wherein said composition is therapeutically effective for infectious diseases, and wherein said packaging material comprises a label or a written material which indicates that said composition can be used for infectious diseases.

11. A commercial package comprising the composition of any of claims 8 and 9, and a written matter associated therewith, wherein the written matter states that the composition can or should be used for preventing or treating infectious diseases.
